(19) United States
(12) Patent Application Publication
(10) Pub. No.: US 2024/0315943 A1
(43) Pub. Date: Sep. 26, 2024

(54) LIGHT-COLORED CATECHOL LIGNOSULFONATE SUNSCREEN MICROCAPSULE, AND PREPARATION THEREFOR AND USE THEREOF

(71) Applicant: South China University of Technology, Guangzhou City (CN)

(72) Inventors: Yong Qian, Guangzhou City (CN); Xueqing Qiu, Guangzhou City (CN); Dongjie Yang, Guangzhou City (CN); Hongming Lou, Guangzhou City (CN); Xinping Ouyang, Guangzhou City (CN)

(21) Appl. No.: 18/291,029

(22) PCT Filed: Jul. 28, 2022
(86) PCT No.: PCT/CN2022/108472

§ 371 Date: Jan. 22, 2024

(30) Foreign Application Priority Data
Jul. 28, 2021 (CN) 202110859121.1

(51) Int. Cl. A61K 8/46; A61K 8/11; A61K 8/35; A61K 8/37; A61Q 17/04

(52) U.S. Cl. CPC A61K 8/466; A61K 8/11; A61K 8/35; A61K 8/37; A61Q 17/04

(57) ABSTRACT

Disclosed in the present invention are a light-colored catechol lignosulfonate sunscreen microcapsule, and a preparation therefor and the use thereof. According to the present invention, lignosulfonate is modified by using a vulcanization method to prepare catechol lignosulfonate, and the catechol lignosulfonate, a sunscreen agent and a surfactant are then subjected to ultrasonic cavitation to prepare a catechol lignosulfonate/chemical sunscreen agent microcapsule emulsion, The preparation process of the lignin/chemical sunscreen agent microcapsule of the present invention is environmentally friendly, has low costs and an excellent ultraviolet absorption performance, has a lighter color compared to other lignin-based sunscreen agents, and has a good biological adhesion and penetration resistance, which solves the problems of poor water resistance, greater pollution caused by the preparation process, easy penetration into the skin to harm the human body, etc. of conventional sunscreen agents.

### FIELD OF THE INVENTION

The present disclosure relates to the field of fine chemicals, and in particular to a light-colored catechol lignosulfonate sunscreen microcapsule and preparation and application thereof.

### BACKGROUND OF THE INVENTION

Light radiation with a wavelength between 10 and 400 nm is internationally referred to as ultraviolet (UV). According to international practice, UV can be divided into UVA band (320 to 400 nm), UVB band (290 to 320 nm), UVC band (200 to 290 nm), and EUV band (10 to 100 nm). Prolonged exposure to UV can cause sunburn, aging, and even skin cancer. EUV and UVC are already absorbed by the ozone layer at high altitudes, while 1 to 10% of UVB and about 90% or more of UVA are still able to reach the earth's surface through the atmosphere. UV in the UVB band has a weak penetrating property, and most of the UV is absorbed by epidermal cells of the human body when irradiated into the human body. However, due to the relatively high UV energy in the UVB band, the UV may cause light damage to the skin, resulting in redness and blisters on the skin. Prolonged exposure can cause skin inflammation, aging, and other adverse reactions, and can cause skin cancer in severe cases. UV in the UVA band has a strong penetrating property and can reach the deep dermis of the human body, thereby causing the deposition of melanin in the human body, and aging and damage to the human skin after a long-term accumulation. Reactive oxygen species (ROS) induced by irradiation with UVA and UVB can damage various cellular components and induce the formation of immunosuppressive cytokines (The FASEB Journal, 2018, 32 (7): 3700-3706). Therefore, the use of UV protectors to avoid UV damage is necessary.

Protective agents can be divided into two major classes, physical sunscreen agents and chemical sunscreen agents, depending on the mechanism of protection. Physical sunscreen agents are relatively heavy, have a poor feel, and are susceptible to photocatalytic reactions. Therefore, chemical sunscreen agents are widely used on the market, and chemical sunscreen additives commonly used on the market include benzophenone-3, xylene, avobenzone, cinnamate, and the like. However, small molecules can be absorbed by the skin after the reaction, and further penetrate the stratum corneum, or enter epidermal cells through follicles, thereby causing allergic reactions and even destroying DNA. In addition, small amounts of chemical and physical sunscreen agents penetrate the skin under UV and generate ROS which cause cell and tissue damage and eventually lead to a series of skin and systemic diseases (Advanced Functional Materials, 2018, 28, 1802127). The problem of penetration of small-molecule sunscreen agents has attracted much attention. Deng et al. have used aldehyde-based hyperbranched poly-glycidyl ethers with bio-adhesive properties as wall materials to encapsulate small-molecule sunscreen agents to form microcapsules, which effectively prevent the problem of penetration of sunscreen agents (Nature Materials, 2015, 14, 1278). However, its UV resistance is limited, and it is difficult to meet the use conditions of harsh environments.

Lignin is abundant in nature, and the content of lignin in natural high molecular compounds is the second and is the highest among natural high molecular polymers containing benzene rings. It protects the internal tissues of the plant from damage caused by UV. This is because, upon irradiation of the plant with UV, the lignin in the plant produces a phenylpropane conjugate, which is effectively shielded from UV. Lignin contains a large number of phenolic hydroxyl groups, benzene rings, carbonyls, methoxies, and other structures, which can effectively absorb UV and scavenge free radicals to play an antioxidant role (Industrial Crops and Products, 2011, 33, 259-276).

While lignin has limited sunscreen properties by itself, there is a synergistic effect with chemical sunscreen agents that can effectively improve the sunscreen performance of commercial sunscreens creams while also improving the photolysis resistance of chemical sunscreen agents (Green Chemistry, 2015, 17: 320-324). Therefore, lignin/sunscreen composite nano-capsules were prepared by coating avobenzone and cinnamate with lignin, and the ratio of lignin to sunscreen agent was adjusted to enhance the synergistic effect of lignin and sunscreen agent. The addition of 10 wt % lignin mixed with blank hand cream resulted in a sunscreen cream with an SPF value of up to 408 and good UV protection within 8 hours (ACS Applied Bio Materials, 2018, 1, 1276).

Nanosized lignin capsules provide a new direction for the development of high-efficiency natural sunscreen creams because of their excellent and long-term UV protection properties. However, lignin microcapsules have a relatively small particle size and little bio-adhesion, so there is still a risk of skin penetration. Therefore, it is necessary to modify lignin to increase the content of phenolic hydroxyl groups in lignin, thereby enhancing the bio-adhesive ability of lignin and lignin microcapsules and improving the safety and use performance of sodium lignosulfonate for UV protective agents.

The existing lignin-based sunscreen creams are mainly prepared by using alkali lignin and enzymatic lignin as raw materials, and the colors of these two lignins are relatively deep. Although lignosulfonate has a lighter color than alkali lignin and enzymatic lignin, the stability of prepared sunscreen agents is inferior to that of general lignin sunscreen agents because lignosulfonate itself easily causes demulsification of the cream, thus limiting its application in the field of sunscreens. In addition, lignosulfonate has a low content of phenolic hydroxyl groups themselves, thus having poor radical scavenging ability and limited absorption in the UV region.

### SUMMARY OF THE INVENTION

### Technical Problems

To address the shortcomings and deficiencies of the prior art, a primary object of the present disclosure is to provide a preparation method for a light-colored catechol lignosulfonate sunscreen microcapsule.

Lignin molecules contain several methoxy groups, which can be modified by a sulfidation method to convert potential methoxies in lignosulfonate into hydroxyl groups by nucleophilic substitution reaction with SO₃²⁻. In the present process, the sulfidation method is improved, and the reaction conditions are relatively milder so that the molecular weight does not significantly decrease during the sulfidation method, which proves that the molecular structure of lignosulfonate itself is not significantly damaged. After demethylation, the phenolic hydroxyl group content of lignosulfonate increases obviously, which results in the structure of catechol and resorcinol in the lignin itself, and improves the bio-adhesive ability of lignosulfonate, so that the problem of poor compatibility between lignosulfonate and frost can be remedied, and stable lignin microcapsules can be formed, which improves the application possibility of lignosulfonate in the field of UV protective agent. Using this process for demethylation, the reaction is carried out in the whole aqueous phase system, the reaction process is relatively more environmentally friendly and green, the reaction cost is less, and the subsequent industrial production has a better application potential.

Another object of the present disclosure is to provide a light-colored catechol lignosulfonate sunscreen microcapsule prepared by the above method.

A further object of the present disclosure is to provide application of the light-colored catechol lignosulfonate sunscreen microcapsule.

### Solutions to Problems

### Technical Solutions

The following technical solutions realize the purpose of the present disclosure.

A preparation method for a light-colored catechol lignosulfonate sunscreen microcapsule includes the following steps:
(1) dissolving lignosulfonate and a sulfidation reagent in an alkali solution, reacting at 40 to 160° C. for 0.1 to 5 hours, dialyzing, and concentrating and drying to obtain catechol lignosulfonate; and
(2) dissolving catechol lignosulfonate in an aqueous solution with a pH of 5 to 9, adding a sunscreen agent and a surfactant, and performing ultrasonic cavitation to obtain a catechol lignosulfonate/chemical sunscreen agent microcapsule emulsion, and obtaining the light-colored catechol lignosulfonate sunscreen microcapsule after centrifugation.

Preferably, in step (1), a weight ratio of lignosulfonate to the sulfidation reagent is 1 to 10:0.2 to 2, more preferably 10:0.7 to 2, and most preferably 8:1 to 5:1.

Preferably, in step (1), the sulfidation reagent is at least one of sulfite, metabisulfite, thiosulfate, and bisulfite.

Preferably, in step (1), lignosulfonate is at least one of sodium lignosulfonate, magnesium lignosulfonate, and calcium lignosulfonate.

More preferably, lignin in lignosulfonate is at least one of bamboo pulp lignosulfonate, wheat straw pulp lignosulfonate, reed lignosulfonate, bagasse pulp lignosulfonate, asparagus pulp lignosulfonate, and cotton pulp lignosulfonate.

The lignin obtained by the treatment and separation of different treatment methods in industry is generally called industrial lignin. Lignin is usually divided and named according to different treatment and separation methods. Different lignins differ greatly in structure, content, and type of active functional groups. Industrial lignin may be divided into four main classes: (1) enzymatic lignin: enzymatic lignin is a kind of lignin obtained after depolymerization and dissolution treatment of lignin raw materials using cellulase and hemicellulose; (2) alkali lignin: alkali lignin mainly comes from alkali pulping waste liquor such as sulfate process and alkyl-alkali process; (3) organic solvent lignin: organic solvent lignin is a class of lignin extracted from plants by organic reagents such as methanol, ethanol, acetone, and the like in a high-temperature environment; and (4) lignosulfonate: lignosulfonate is derived from sulfite pulping waste liquor, which has good water solubility and broad application prospects.

Preferably, in step (1), the alkali solution is a 1 to 20 wt % sodium hydroxide solution, more preferably a 1.5 to 15 wt % sodium hydroxide solution, and most preferably a 5 to 15 wt % sodium hydroxide solution; a weight ratio of lignosulfonate to the alkali solution is 1 to 10:30.

Preferably, in step (1), the reacting is performed at 70 to 140° C. for 1 to 2.5 hours.

Preferably, in step (1), the dialyzing is performed for 2 to 8 days and more preferably 4 to 6 days with a dialysate of water.

Preferably, in step (2), a ratio of a total weight of a solution obtained after dissolving catechol lignosulfonate in the aqueous solution with the pH of 5 to 9 to a weight of the sunscreen agent is 1:1 to 10:1, more preferably 1:1 to 4:1, and most preferably 7:3.

Preferably, in step (2), the surfactant is added in an amount of 1 to 10 wt % of a total weight of catechol lignosulfonate, the aqueous solution with a pH of 5 to 9, and the sunscreen agent, and more preferably 3 to 10 wt %.

Preferably, in step (2), a weight ratio of catechol lignosulfonate to the aqueous solution with the pH of 5 to 9 is 1 to 10:20 to 100, and more preferably 1:10 to 33.

Preferably, in step (2), the pH of the solution obtained after dissolving catechol lignosulfonate in the aqueous solution should be in a range of 6 to 8.

Preferably, in step (2), the sunscreen agent is at least one of UVA and UVB type sunscreen agents, and more preferably at least one of avobenzone, ethylhexyl methoxycinnamate, homosalate, and oxybenzone; when more than one of the sunscreen agents is used, good miscibility is required.

Preferably, in step (2), the surfactant is at least one of Tween, alkyl polyglycoside, and sucrose ester.

Preferably, in step (2), the ultrasonic cavitation is performed for 1 to 20 minutes with a power of 200 to 1500 W, and more preferably for 3 to 10 minutes with a power of 400 to 1000 W.

Preferably, in step (2), the catechol lignosulfonate/chemical sunscreen agent microcapsule emulsion may be washed by centrifugation to remove excess catechol lignosulfonate to obtain the catechol lignosulfonate/chemical sunscreen agent microcapsule.

More preferably, the centrifugation rate is 5000 to 15000 r/min and the time of centrifugation and washing is 20 to 40 minutes.

The present disclosure provides a light-colored catechol lignosulfonate sunscreen microcapsule prepared by the above method.

The light-colored catechol lignosulfonate sunscreen microcapsule of the present disclosure has an amphiphilic spherical structure with a particle size of 100 to 1000 nm, excellent UV absorption performance, good stability, and at the same time, excellent water resistance and permeation resistance.

The present disclosure provides application of the light-colored catechol lignosulfonate sunscreen microcapsule in the field of chemical sunscreen. The chemical sunscreen does not take a living body as the sunscreen object.

More preferred is application in the preparation of sunscreen products.

Lignin is a natural macromolecular UV protective agent in plants, which has good UV absorption and antioxidant function and has good biocompatibility. The demethylated lignin after chemical modification contains more phenolic hydroxyl groups, and the crosslinking reaction occurs more easily when the chemical sunscreen agents are encapsulated by ultrasonic cavitation. The size of microcapsules prepared by the dispersing oil phase will be nano-scale when the surfactant is added, and the UV scattering effect can be enhanced. On this basis, the structure of catechol and resorcinol distributed on the surface of the bio-adhesive microcapsules of catechol lignosulfonate/chemical sunscreen agent endows the microcapsules with good water resistance and permeation resistance, improving the use efficiency and safety. The preparation process of bio-adhesive microcapsules of catechol lignosulfonate/chemical sunscreen agents is environment-friendly, safe, and efficient, which promotes the application of renewable resources lignin in the daily chemical field and solves the problem of insufficient crosslinking ability of lignosulfonate, and has broad application prospects.

### Beneficial Effects of the Disclosure

### Beneficial Effects

Compared to the prior art, the present disclosure has the following advantages and beneficial effects:
(1) Lignosulfonate has good UV and antioxidant properties and good biocompatibility. There are many phenolic hydroxyl active sites in the molecule after chemical modification, which can make use of free radical crosslinking in the ultrasonic cavitation process. It can effectively encapsulate small-molecule chemical sunscreen agents for synergistic sunscreens, improve the photolysis resistance of small-molecule chemical sunscreen agents, and achieve durable sunscreens.
(2) The lignosulfonate demethylation process is green and environment-friendly, the whole reaction process is carried out in the water phase, no organic solvent is used, the reaction process is safe and environment-friendly, and the reaction process is simple, easy to achieve large-scale preparation, with good industrial prospects.
(3) Compared with the previous sulfurization demethylation methods, the reaction conditions used in the specification are milder, and the preparation cost is lower. The sulfidation reagents used in the specification are all inorganic sulfides, which are more green and environmentally friendly. The methyl group in lignosulfonate can be removed at a lower cost, which effectively promotes the construction of catechol lignosulfonate.
(4) The structure of catechol and resorcinol distributed on the surface of the bio-adhesive microcapsule of catechol lignosulfonate/chemical sunscreen agent provides the microcapsule with good adhesion and penetration resistance, improves the use efficiency and safety, solves the problems of poor water resistance of traditional sunscreen agents and being easy to penetrate the skin and damage the human body, and achieves safe and efficient sunscreen skin care.

### BRIEF DESCRIPTION OF THE DRAWINGS

FIG. 1 is a bio-adhesive microcapsule emulsion of a catechol lignosulfonate/chemical sunscreen agent obtained by ultrasonic cavitation in step (2) of embodiment 1;

FIG. 2 is a picture of original sodium lignosulfonate (left) and catechol sodium lignosulfonate prepared in embodiment 1 (right) when subjected to a Folin-phenol test;

FIG. 3 is a UV spectrum of bio-adhesive microcapsule sunscreen creams of a catechol lignosulfonate/chemical sunscreen agent and lignosulfonate/chemical sunscreen agent microcapsule sunscreen creams in a range of 260 to 400 nm obtained in embodiment 1;

FIG. 4 is a graph of laser confocal testing of bio-adhesive microcapsule sunscreen creams of a catechol lignosulfonate/chemical sunscreen agent and sodium lignosulfonate/chemical sunscreen agent microcapsule sunscreen creams obtained in embodiment 1;

FIG. 5 is data relating to particle size testing of bio-adhesive microcapsules of a catechol lignosulfonate/chemical sunscreen agent and a sodium lignosulfonate/chemical sunscreen agent microcapsule obtained in embodiment 1; and

FIG. 6 is an ultrasound-cavitated emulsion of catechol lignosulfonate obtained in comparative embodiment 1.

### DETAILED DESCRIPTION OF THE EMBODIMENTS

The present disclosure is further described in detail in combination with embodiments and drawings below, but implementations of the present disclosure are not limited herein.

The specific conditions not specified in the embodiments of the present disclosure are performed according to conventional conditions or conditions suggested by the manufacturer. The raw materials, reagents, and the like not specified by the manufacturer are conventional products that can be obtained through commercial purchase.

### Embodiment 1

(1) 10 g of sodium lignosulfonate and 1.5 g of sodium sulfite were dissolved in 30 ml of 10 wt % NaOH solution, and a condensation reflux reaction was performed at 90° C. for 1 hour. After the reaction was completed, the solution was cooled to room temperature, and the reaction solution was subjected to dialysis purification treatment in pure water. After the dialysis, the sample solution was concentrated and dried to obtain catechol lignosulfonate solid powder.
(2) 0.21 g of catechol lignosulfonate was dissolved in 6.79 g of ultrapure water to prepare a 5 wt % solution, 3 g of a mixture of ethylhexyl methoxycinnamate and avobenzone (a mass ratio of 4:1) and 0.7 g of Tween were added, ultrasonic cavitation was performed at an ultrasonic power of 400 W for 3 minutes to obtain a bio-adhesive microcapsule emulsion of a catechol lignosulfonate/chemical sunscreen agent before being centrifuged at 10000 r/min for 30 minutes to obtain a bio-adhesive microcapsule of a catechol lignosulfonate/chemical sunscreen agent. The bio-adhesive microcapsule sunscreen creams of the catechol lignosulfonate/chemical sunscreen agent were prepared by mixing the microcapsule with a blank cream (NIVEA deep moist hand cream, NIVEA (Shanghai) Co., Ltd.) without sunscreen active ingredients at a mass ratio of 1:9.

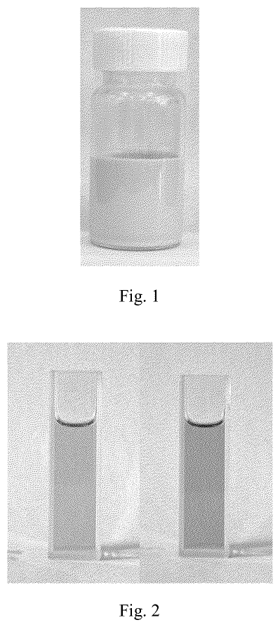

FIG. 1 is an ultrasound-cavitated emulsion of a catechol lignosulfonate/chemical sunscreen agent prepared in step (2). The ultrasonic cavitation emulsion obtained after demethylation has good stability.

FIG. 2 is a picture of original sodium lignosulfonate and catechol lignosulfonate of step (1) when subjected to the Folin-phenol test. On the left side, the absorption value of original sodium lignosulfonate at 760 nm was 0.216, and the absorption value of the modified sodium lignosulfonate at 760 nm reached 0.3739. The phenolic hydroxyl content of the sodium lignosulfonate increased from 0.631 mmol/g to 1.234 mmol/g, which increased by 95.5%. It can also be seen from the drawings that the color of the sodium lignosulfonate test solution after the demethylation reaction was darker, which also indicated that the phenolic hydroxyl content of the sodium lignosulfonate increased.

FIG. 3 is a graph showing the UV spectrum in the range of 290 to 400 nm of the bio-adhesive microcapsule sunscreen creams of a catechol lignosulfonate/chemical sunscreen agent obtained in the present embodiment 1 and the sodium lignosulfonate/chemical sunscreen agent microcapsule sunscreen creams prepared by replacing catechol lignosulfonate in step 2 of the embodiment 1 with sodium lignosulfonate measured by Shimadzu UV-2600 UV-visible spectrophotometer; it can be seen therefrom that The UV transmittance of the bio-adhesive microcapsule sunscreen cream of the catechol lignosulfonate/chemical sunscreen agent was significantly lower than that of the sodium lignosulfonate/chemical sunscreen agent microcapsule sunscreen creams, indicating that more UV could be blocked. In terms of UV sun protection factor (SPF), the bio-adhesive microcapsule sunscreen creams of the catechol lignosulfonate/chemical sunscreen agent have an SPF value of 116, whereas the sodium lignosulfonate/chemical sunscreen agent microcapsule sunscreen creams have an SPF value of only 39.

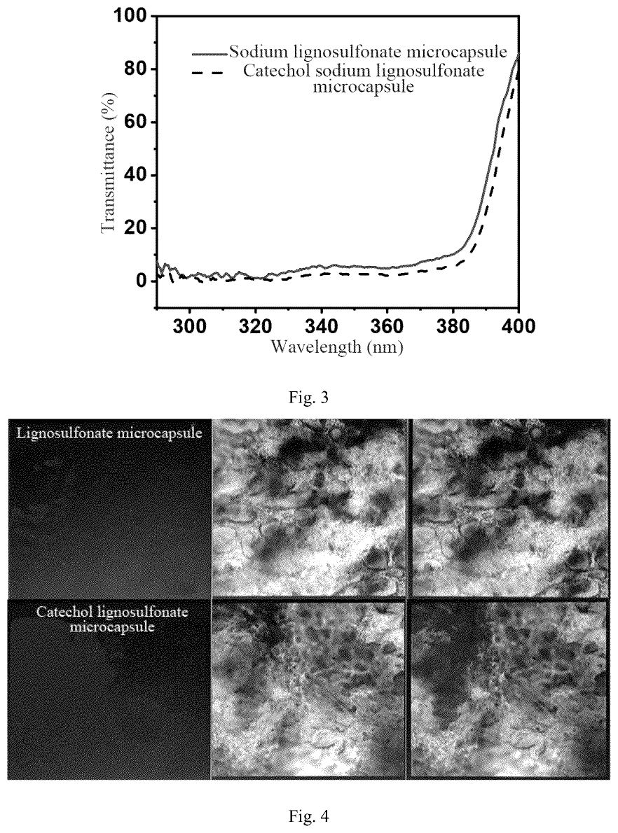

FIG. 4 is a graph of laser confocal testing of bio-adhesive microcapsule sunscreen creams of a catechol lignosulfonate/chemical sunscreen agent and sodium lignosulfonate/chemical sunscreen agent microcapsule sunscreen creams obtained in embodiment 1; Laser confocal microscopy was used to test the adhesion of bio-adhesive microcapsule sunscreen creams of a catechol lignosulfonate/chemical sunscreen agent and sodium lignosulfonate/chemical sunscreen agent microcapsule sunscreen creams to the skin. The sodium lignosulfonate/chemical sunscreen agent microcapsule sunscreen creams prepared by replacing catechol lignosulfonate in step 2 of embodiment 1 with original sodium lignosulfonate have poor adhesion to the skin, and the blue fluorescence signal of lignin on the surface is weak, demonstrating that the microcapsules adhering to the skin surface are washed away, while the bio-adhesive microcapsule sunscreen creams of catechol lignosulfonate/chemical sunscreen agent can still maintain a strong fluorescence signal after washing, demonstrating that the phenol hydroxyl on the surface of sodium lignosulfonate increases after demethylation compared with original sodium lignosulfonate, and the adhesion performance is significantly improved.

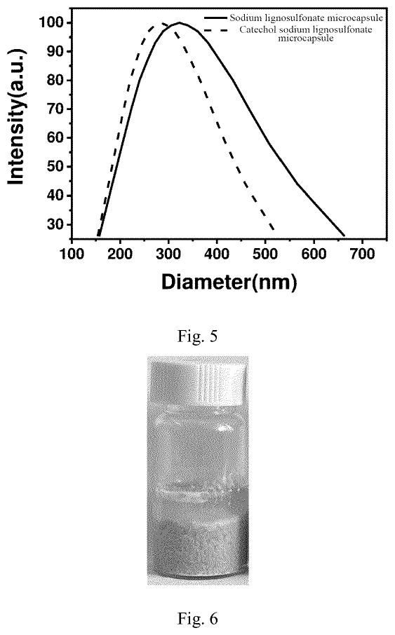

FIG. 5 is data relating to particle size testing of the bio-adhesive microcapsules of a catechol lignosulfonate/chemical sunscreen agent obtained in the present embodiment 1 tested by the Malvern laser particle analyzer. The size of microcapsules has a great influence on their storage stability at room temperature, and the larger the particle size, the more difficult it is to store. It can be seen from the drawings that the average particle size of the bio-adhesive microcapsules of the catechol lignosulfonate/chemical sunscreen agent is about 300 nm, while the median particle size of the sodium lignosulfonate/chemical sunscreen agent microcapsule is 350 nm, and the particle size distribution of the bio-adhesive microcapsules of the catechol lignosulfonate/chemical sunscreen agent is narrower, demonstrating that the product obtained after the demethylation treatment has better homogeneity. Therefore, the preparation method of the present disclosure can significantly improve the storage time and stability of microcapsules.

### Embodiment 2

(1) 10 g of magnesium lignosulfonate and 2 g of sodium sulfite were dissolved in 30 mL of 15 wt % NaOH solution and subjected to a condensation reflux reaction at 120° C. for 1 hour. After the reaction was completed, the solution was cooled to room temperature, and the reaction solution was subjected to dialysis purification treatment in pure water. After the dialysis, the sample solution was concentrated and dried to obtain the catechol lignosulfonate solid powder.
(2) 0.21 g of catechol lignosulfonate was dissolved in 6.79 g of ultrapure water to prepare a 3 wt % solution, 4 g of a mixture of homosalate and avobenzone (a mass ratio of 4:1) and 1 g of Tween were added, ultrasonic cavitation was performed at an ultrasonic power of 600 W for 5 minutes, and the solution was centrifuged at 8000 r/min for 30 minutes to obtain the bio-adhesive microcapsule of the catechol lignosulfonate/chemical sunscreen agent. The bio-adhesive microcapsule sunscreen creams of the catechol lignosulfonate/chemical sunscreen agent were prepared by mixing the microcapsule with a blank cream (NIVEA deep moist hand cream, NIVEA (Shanghai) Co., Ltd.) without sunscreen active ingredients at a mass ratio of 1:9.

The same microcapsule preparation process, UV-visible spectrum analysis, laser confocal test, and particle size test as in embodiment 1 were used. The results were substantially the same as in FIGS. 1, 2, 3, 4, and 5. The bio-adhesive microcapsule sunscreen creams of the catechol lignosulfonate/chemical sunscreen agent had an SPF value of 98.

### Embodiment 3

(1) 10 g of sodium lignosulfonate and 0.7 g of sodium metabisulfite were dissolved in 40 mL of 5 wt % NaOH solution, and the condensation reflux reaction was performed at 70° C. for 2.5 hours. After the reaction was completed, the solution was cooled to room temperature, and the reaction solution was subjected to dialysis purification treatment in pure water. After the dialysis, the sample solution was concentrated and dried to obtain the catechol lignosulfonate solid powder.
(2) 0.6 g of catechol lignosulfonate was dissolved in 6.4 g of ultrapure water to prepare a 3 wt % solution, 3 g of a mixture of ethylhexyl methoxycinnamate and avobenzone (a mass ratio of 4:1) and 0.3 g of sucrose ester were added, ultrasonic cavitation was performed at an ultrasonic power of 400 W for 3 minutes, and centrifugation was performed at 5000 r/min for 40 minutes to obtain the bio-adhesive microcapsule of the catechol lignosulfonate/chemical sunscreen agent. The bio-adhesive microcapsule sunscreen creams of the catechol lignosulfonate/chemical sunscreen agent were prepared by mixing the microcapsule with a blank cream (NIVEA deep moist hand cream, NIVEA (Shanghai) Co., Ltd.) without sunscreen active ingredients at a mass ratio of 1:9.

The same microcapsule preparation process, UV-visible spectrum analysis, laser confocal test, and particle size test as in embodiment 1 were used. The results were substantially the same as in FIGS. 1, 2, 3, 4, and 5. The bio-adhesive microcapsule sunscreen creams of the catechol lignosulfonate/chemical sunscreen agent had an SPF value of 104.

### Embodiment 4

(1) 7 g of calcium lignosulfonate and 1.5 g of sodium thiosulfate were dissolved in 20 mL of 2.5 wt % NaOH solution, and a condensation reflux reaction was performed at 140° C. for 1.5 hours. After the reaction was completed, the solution was cooled to room temperature, and the reaction solution was subjected to dialysis purification treatment in pure water. After the dialysis, the sample solution was concentrated and dried to obtain the catechol lignosulfonate solid powder.
(2) 0.21 g of catechol lignosulfonate was dissolved in 6.79 g of ultrapure water to prepare a 3 wt % solution, 3 g of a mixture of homosalate and avobenzone (a mass ratio of 4:1) and 0.4 g of alkyl polyglycoside were added, ultrasonic cavitation was performed at an ultrasonic power of 700 W for 5 minutes, and the centrifugation was performed at 10000 r/min for 30 minutes to obtain the bio-adhesive microcapsule of the catechol lignosulfonate/chemical sunscreen agent. The bio-adhesive microcapsule sunscreen creams of the catechol lignosulfonate/chemical sunscreen agent were prepared by mixing the microcapsule with a blank cream (NIVEA deep moist hand cream, NIVEA (Shanghai) Co., Ltd.) without sunscreen active ingredients at a mass ratio of 1:9.

The same microcapsule preparation process, UV-visible spectrum analysis, laser confocal test, and particle size test as in embodiment 1 were used. The results were substantially the same as in FIGS. 1, 2, 3, 4, and 5. The bio-adhesive microcapsule sunscreen creams of the catechol lignosulfonate/chemical sunscreen agent had an SPF value of 113.

### Comparative Embodiment 1

(1) 10 g of sodium lignosulfonate was dissolved in 30 ml of 10 wt % NaOH solution, and a condensation reflux reaction was performed at 90° C. for 1 hour under a nitrogen atmosphere. After the reaction was completed, the solution was cooled to room temperature, and the reaction solution was subjected to dialysis purification treatment in pure water. After the dialysis, the sample solution was concentrated and dried to obtain a catechol lignosulfonate solid powder.
(2) 0.21 g of catechol lignosulfonate was dissolved in 6.79 g of ultrapure water to prepare a 5 wt % solution, 3 g of a mixture of ethylhexyl methoxycinnamate and avobenzone (a mass ratio of 4:1) and 0.7 g of Tween were added, and ultrasonic cavitation was performed at an ultrasonic power of 400 W for 3 minutes. A sodium lignosulfonate/chemical sunscreen agent ultrasonic cavitation emulsion was obtained.

FIG. 6 is a catechol lignosulfonate ultrasound-cavitated emulsion obtained in the present comparative embodiment. It can be seen from the drawings that the obtained ultrasonic cavitation emulsion had an obvious phenomenon of stratification. The reason was that the proper amount of surfactant reduced the interfacial tension between water and oil, increased the specific surface area of oil droplets, and facilitated the adsorption and crosslinking of sodium lignosulfonate on the surface of oil droplets. However, the addition of excess surfactant can cause the interaction of excess surfactant itself, resulting in the polymerization of droplets, thus making the particle size of the prepared microcapsules too large, and unstable and then breaking the emulsion.

When the microcapsules were prepared by subsequently adding a chemical sunscreen agent and a surfactant, the microcapsules could not be prepared basically or the prepared microcapsules were easily broken. The results also showed that the stability of modified lignosulfonate in the preparation of microcapsules was improved obviously after the treatment of the sulfidation method.

The above embodiments are the better embodiments of the present disclosure, but the implementations of the present disclosure are not limited by the above embodiments, and any other changes, modifications, substitutions, combinations, and simplifications that do not deviate from the spirit and principle of the present disclosure shall be equivalent replacement modes and shall be included in the scope of protection of the present disclosure.

1. A preparation method for a light-colored catechol lignosulfonate sunscreen microcapsule, comprising the following steps:
(1) dissolving lignosulfonate and a sulfidation reagent in an alkali solution, reacting at 40 to 160° C. for 0.1 to 5 hours, dialyzing, and concentrating and drying to obtain catechol lignosulfonate; and
(2) dissolving catechol lignosulfonate in an aqueous solution with a pH of 5 to 9, adding a sunscreen agent and a surfactant, and performing ultrasonic cavitation to obtain a catechol lignosulfonate/chemical sunscreen agent microcapsule emulsion, and obtaining the light-colored catechol lignosulfonate sunscreen microcapsule after centrifugation, wherein
in step (1), a weight ratio of lignosulfonate to the sulfidation reagent is 1 to 10:0.2 to 2; the sulfidation reagent is at least one of sulfite, metabisulfite, thiosulfate, and bisulfite;
in step (2), a ratio of a total weight of a solution obtained after dissolving catechol lignosulfonate in the aqueous solution with the pH of 5 to 9 to a weight of the sunscreen agent is 1:1 to 10:1;
in step (2), the surfactant is added in an amount of 1 to 10 wt % of a total weight of catechol lignosulfonate, the aqueous solution with a pH of 5 to 9, and the sunscreen agent;
in step (2), a weight ratio of catechol lignosulfonate to the aqueous solution with the pH of 5 to 9 is 1 to 10:20 to 100;
in step (2), the sunscreen agent is at least one of avobenzone, ethylhexyl methoxycinnamate, homosalate, and oxybenzone; and
in step (2), the surfactant is at least one of Tween, alkyl polyglycoside, and sucrose ester.

2. The preparation method for a light-colored catechol lignosulfonate sunscreen microcapsule according to claim 1, wherein in step (1), the weight ratio of lignosulfonate to the sulfidation reagent is 10:0.7 to 2.

3. The preparation method for a light-colored catechol lignosulfonate sunscreen microcapsule according to claim 1, wherein in step (2), a ratio of the total weight of the solution obtained after dissolving catechol lignosulfonate in the aqueous solution with the pH of 5 to 9 to the weight of the sunscreen agent is 1:1 to 4:1;
in step (2), the surfactant is added in an amount of 3 to 10 wt % of the total weight of catechol lignosulfonate, the aqueous solution with the pH of 5 to 9, and the sunscreen agent; and
in step (2), a weight ratio of catechol lignosulfonate to the aqueous solution with the pH of 5 to 9 is 1:10 to 33.

4. The preparation method for a light-colored catechol lignosulfonate sunscreen microcapsule according to claim 1, wherein in step (1), lignosulfonate is at least one of sodium lignosulfonate, magnesium lignosulfonate, and calcium lignosulfonate; lignin in lignosulfonate is at least one of bamboo pulp lignosulfonate, wheat straw pulp lignosulfonate, reed lignosulfonate, bagasse pulp lignosulfonate, asparagus pulp lignosulfonate, and cotton pulp lignosulfonate.

5. The preparation method for a light-colored catechol lignosulfonate sunscreen microcapsule according to claim 1, wherein in step (1), the reacting is performed at 70 to 140° C. for 1 to 2.5 hours; and
in step (2), the ultrasonic cavitation is performed for 1 to 20 minutes with a power of 200 to 1500 W.

6. The preparation method for a light-colored catechol lignosulfonate sunscreen microcapsule according to claim 1, wherein in step (1), the alkali solution is a 1 to 20 wt % sodium hydroxide solution, and a weight ratio of lignosulfonate to the alkali solution is 1 to 10:30; in step (1), the dialyzing is performed for 2 to 8 days with a dialysate of water.

7. A light-colored catechol lignosulfonate sunscreen microcapsule obtained by the preparation method according to claim 1.

8. Application of the light-colored catechol lignosulfonate sunscreen microcapsule according to claim 7 in preparation of a sunscreen product.

9. A light-colored catechol lignosulfonate sunscreen microcapsule obtained by the preparation method according to 2.

10. A light-colored catechol lignosulfonate sunscreen microcapsule obtained by the preparation method according to 3.

11. A light-colored catechol lignosulfonate sunscreen microcapsule obtained by the preparation method according to 4.

12. A light-colored catechol lignosulfonate sunscreen microcapsule obtained by the preparation method according to 5.

13. A light-colored catechol lignosulfonate sunscreen microcapsule obtained by the preparation method according to 6.

14. Application of the light-colored catechol lignosulfonate sunscreen microcapsule according to claim 9 in preparation of a sunscreen product.

15. Application of the light-colored catechol lignosulfonate sunscreen microcapsule according to claim 10 in preparation of a sunscreen product.

16. Application of the light-colored catechol lignosulfonate sunscreen microcapsule according to claim 11 in preparation of a sunscreen product.

17. Application of the light-colored catechol lignosulfonate sunscreen microcapsule according to claim 12 in preparation of a sunscreen product.

18. Application of the light-colored catechol lignosulfonate sunscreen microcapsule according to claim 13 in preparation of a sunscreen product.
